(19) United States
(12) Patent Application Publication
(10) Pub. No.: US 2015/0038904 A1
(43) Pub. Date: Feb. 5, 2015

(54) AUTO-DISABLE SYRINGE ASSEMBLY

(71) Applicant: Wolfe Tory Medical, Inc., Salt Lake City, UT (US)

(72) Inventors: Marshall T. Denton, Salt Lake City, UT (US); Perry W. Croll, Salt Lake City, UT (US); Mark A. Christensen, Salt Lake City, UT (US); Huy N. Tran, Riverton, UT (US)

(73) Assignee: Wolfe Tory Medical, Inc., Salt Lake City, UT (US)

(21) Appl. No.: 14/386,284

(22) PCT Filed: Mar. 22, 2013
(86) PCT No.: PCT/IB2013/052303

§ 371 Date: Sep. 18, 2014

Related U.S. Application Data
(60) Provisional application No. 61/614,996, filed on Mar. 23, 2012.

(51) Int. Cl. A61M 5/50; A61M 5/315

(52) U.S. Cl. CPC A61M 5/5066; A61M 5/31501
USPC 604/110; 604/218

(57) ABSTRACT

A syringe structured to permit multiple filling and emptying cycles with a single syringe, but then to automatically disable that syringe to resist syringe reuse subsequent to a final dose-ejecting cycle. The syringe includes a plunger with a distal tip carrying capture structure that protrudes from the dispensing aperture of the syringe at a fluid fully-expelled position. A fluid discharge attachment can be coupled to the syringe to guide dispensing one or more dose of treatment substance. A first locking element, typically carried by the fluid discharge attachment, couples with the capture structure to resist retraction of the fully-depressed plunger, and thereby resists reciprocation of the plunger to prevent reuse of the syringe. Effectively, the discharge aperture of the syringe is pinned between an internally disposed stopper and the external capture structure. A stem of the plunger may optionally be structured to detach, leaving the stopper behind inside the syringe body, to further frustrate reuse of the syringe. Sometimes, a second locking element may be included to also resist unscrewing a fluid discharge device from a syringe.

### BACKGROUND OF THE INVENTION

1. The Field of the Invention

The present invention relates to devices adapted to control motion of fluid. in particular, preferred embodiments are adapted to permit multiple filling and emptying cycles with a single syringe, but then automatically disable that syringe to resist syringe reuse subsequent to a final dose-ejecting cycle.

2. Related Art

Syringes adapted to permit only a single use are known. Desirably, after a single use, such a syringe will automatically be rendered inoperable. Such auto-disable syringes are desirable, for example, to reduce spread of blood-borne disease between individuals who share a common needle.

A plurality of differently structured syringes have been developed to address the desire for an effective auto-disable syringe. A problem with many commercially available syringes is that they do not permit use of a single syringe to permit multiple filling and complete emptying cycles, but then automatically disable that syringe to resist syringe reuse subsequent to a final dose-ejecting cycle. There is a need for a syringe that can be coupled to a needle to inspire fluid into the syringe, then expel that fluid into a container to reconstitute a treatment substance. After the treatment substance is reconstituted, it is desirable to refill the same syringe with one or more dose of treatment substance. It is further desirable for the syringe to then auto-disable upon dispensing the entire contents of the syringe. Sometimes, it is desirable to detach a needle used to inspire fluids into the syringe and to attach a different fluid-guiding element to the syringe before dispensing the fluid contents of the syringe in an auto-disable cycle.

As a practical matter, a syringe should first be completely emptied before inspiring one or more dose of reconstituted treatment substance, to avoid dilution of the treatment substance to an unknown and variable degree. It is not feasible to substantially empty certain commercially available auto-disable syringes without risk of accidentally engaging their auto-disable feature, consequently requiring use of a different syringe to administer the treatment substance.

In United States patent application No. 2010/0030146, Kakish et al. disclose an auto-disable device for syringes that permits a user to fill the syringe by inspiring fluid through a needle, and automatically disable that syringe upon discharge of the syringe contents. Their device's needle is not removable after being coupled to the syringe body, and consequently, their syringe assembly cannot realistically be used to reconstitute a treatment substance and subsequently dispense a portion of that treatment substance with the same syringe. Once fluid begins to be expelled from their syringe, an internal mechanism is displaced to prevent fluid from being inspired into the syringe. Therefore, refilling the same syringe is precluded.

In U.S. Pat. No. 6,752,784, Tsai discloses a safety syringe of the type in which capture structure carried on a plunger is coupled with locking structure to maintain a blocking element inside the syringe discharge aperture. The plunger inevitably couples with capture structure upon complete discharge of fluid contents from the syringe. Reuse of the syringe is consequently resisted subsequent to complete discharge of the syringe. In fact, attempting to retract his plunger from a fully-expelled position causes the distal portion of the syringe barrel to retract into his syringe body, which can serve as a safety cover for a needle assembly. Similar devices are disclosed in United States patent application No. 2006/0084915 to Chang, and United States patent application No. 2009/0247948 to Walsh et al.

A commercially available auto-disable syringe includes a 1 mL Auto Disable Syringe, available from Zhejiang Yusheng Medical Instrument Co., Ltd, having a place of business in Xuanmen Industry Zone, Yuhuan City, Zheijiang, China, and a world wide web address of zjyusheng.com. Such syringe includes a plunger having distally disposed capture structure on a shaft that couples with locking structure in the syringe discharge luer effective to resist retraction of the plunger from a fluid fully-expelled position. If a user retracts the plunger stem with sufficient force, a proximal portion of the plunger stem is structured to break off and separate, leaving behind a stopper that is essentially pinned inside the discharge aperture of the syringe, thereby resisting reuse of the syringe.

### BRIEF SUMMARY

The invention may be embodied to provide an apparatus and a method of use of that apparatus. A currently preferred apparatus forms an assembly including a syringe body extending from an open proximal end to a discharge aperture at a distal end. A plunger assembly is disposed for reciprocal motion inside the body between a dose-loaded position and a dose fully-expelled position. A distal end of the plunger assembly carries capture structure configured to protrude distally, by an operable distance, from the syringe discharge aperture when the plunger assembly is disposed at the fully-expelled position.

The aforementioned assembly also includes a first locking element that is separate and discrete from the syringe body and is disposable at a position distal to the distal end of the syringe body. The first locking element is configured and arranged to couple with capture structure, when the plunger is displaced distally to the fully-expelled position, effective to resist reuse of the syringe. In most cases, a first locking element is affixed to an end-use fluid-dispensing or fluid-acquiring attachment that can be coupled to be syringe discharge aperture to permit one final ejection of fluid through the attachment by operation of the plunger. The first locking element can be an integral part of a fluid-guiding device, such as a fluid-dispensing or fluid-acquiring attachment, or may be temporarily, or permanently, coupled to the attachment. A first locking element may also be embodied as a separate, stand-alone component.

A currently preferred first locking element is structured to effect a press-fit engagement, or friction coupling, with a fluid-guiding device. In such an arrangement, attempting to disengage a fluid-guiding device from a syringe body (e.g. by unscrewing a conventional luer-locking joint) subsequent to effecting a captured plunger coupling with a locking element will simply disengage the first locking element from its press-fit engagement with the fluid-guiding device. The disengaged first locking element remains coupled to the capture structure, and the syringe is disabled to resist plunger reciprocation and syringe reuse. Essentially, the press-fit coupling desirably forms a “weakest link” to prevent the plunger from breaking at a location that would permit reuse of the syringe.

An operable plunger assembly may sometimes include decoupling structure configured to permit separation of a proximal stem end of the plunger assembly from the distal end of the plunger assembly responsive to a user attempting to proximally displace the plunger assembly from a captured configuration comprising the fully-expelled position. An exemplary decoupling structure includes a pull-apart joint disposed at an intermediate location of the plunger assembly. One operable pull-apart joint includes a first interface structured to fail, or otherwise separate, in shear under a lower stem tension load than required for a critical cross-section of capture structure to fail under tension. The currently preferred pull-apart joint includes a one-way pull-apart joint with mutually cooperating surfaces of the plunger assembly being structured to bear in compression as the plunger is displaced from a dose-loaded position toward the dose fully-expelled position.

Typically, a distal end of the syringe body carries a first length of hollow male luer-locking taper terminating at a distal interface. In such case, a proximal end of a first locking element may be configured and arranged to cause a structural interference with the distal interface effective to resist proximal displacement of capture structure carried by the plunger. Sometimes a first locking element may be structured as a second length of hollow male luer-locking taper sized as an operable extension of the first length.

One operable capture structure includes a proximally facing surface. Upon coupling with the capture structure, locking structure of a cooperating first locking element is then configured and arranged to cause a structural interference with the proximally facing surface. An operable first locking structure can include a self-biased finger. Similarly, locking structure of a first locking element can include a plurality of self-biased fingers that are displaceable by proximal movement of capture structure to form a friction contact interface between the fingers and capture structure.

Sometimes certain embodiments of the invention may include a second locking element structured to resist removal of an end-use fluid-dispensing device from captured engagement with a syringe. An operable second locking element includes structure carried by the end-use fluid-dispensing device which automatically engages with, and creates a structural interference with a female thread of a luer-lock device to resist rotation of the end-use fluid-dispensing device in a disassembling direction.

A method of use of a preferred apparatus includes providing an auto-disable syringe having a plunger disposed for reciprocation in a body effective to inspire and eject fluid. A user may couple a fluid-guiding or -inspiring attachment (e.g. a needle) to the syringe, or use the syringe a stand-alone device to inspire a dose of fluid into the syringe. After a dose of fluid is inspired into the syringe, the user may then inject at least a portion of the dose of fluid into a container effective to reconstitute a treatment substance. A needle can be coupled to the syringe discharge orifice to facilitate guiding discharged fluid into a container, such as a vial. Typically, the user will displace the plunger to a fluid fully-expelled position in preparation for loading one or more dose of treatment substance into the syringe. The user may then inspire a portion of the moment substance into the syringe. Finally, the user will distally displace the plunger to the fluid fully-expelled position to expel a final dose of treatment substance while simultaneously coupling a first locking element to the plunger effective to resist retraction of the plunger from the fully-expelled position.

After inspiring a portion of treatment substance into the syringe, the user may remove the fluid-inspiring device and couple a second, and different, fluid-dispensing device to the syringe. The second fluid dispensing device may sometimes be made reference to as an end-use device. In that case, the second fluid-dispensing device typically carries the first locking element, which is structured to couple with capture structure carried by the plunger. Conventionally, capture structure is configured to protrude distally from a discharge aperture of the syringe when the plunger is disposed at the fluid fully-expelled position. Sometimes, subsequent to coupling the first locking element to the plunger, a user may proximally retract a stem portion of the plunger effective to separate the stem portion from a distal portion of the plunger. Sometimes, assembling the second fluid-dispensing device to a luer-lock device carried by the syringe automatically engages a second locking element effective to resist removal of the second fluid-dispensing device from the syringe.

### BRIEF DESCRIPTION OF THE DRAWINGS

In the drawings, which illustrate what are currently regarded as the best modes for carrying out the invention, and in which similar components are generally designated with similar numerals:

FIG. 1 is an exploded assembly plan view, to scale, of a currently preferred embodiment that is constructed according to certain principles of the instant invention;

FIG. 2 is a side view, partially in section, of a portion of the plunger illustrated in FIG. 1;

FIG. 3 is a side view, partially in section, of a portion of the plunger illustrated in FIG. 2, at a further state of assembly;

FIG. 4 is a side view in elevation of a portion of the embodiment illustrated in FIG. 1, at a dose-loaded position;

FIG. 5 is a side view in elevation of the embodiment in FIG. 4, but at a fully-expelled position;

FIG. 6 is a side view in elevation of the embodiment in FIG. 4, but in a frustrated-reuse position;

FIG. 7 is a side view in elevation of an embodiment structured according to certain principles of the invention, in combination with a plurality of operable attachments, partially exploded;

FIG. 8 is a close-up side view in elevation and cross-section of a portion of a currently preferred embodiment that is constructed according to certain principles of the instant invention, at a dose-expelled and captured position;

FIG. 9 is a close-up side view in elevation and partial cross-section of a portion of an alternative embodiment that is constructed according to certain principles of the instant invention, at a fully-expelled position;

FIG. 10 is a close-up side view in elevation and partial cross-section of the embodiment in FIG. 9, but at a fully-expelled and captured position;

FIG. 11 is a perspective cross-section view looking at the proximal end of a currently preferred first looking element;

FIG. 12 is a view in perspective of a currently preferred second locking element;

FIG. 13 is a proximal end view of the embodiment in FIG. 12;

FIG. 14 is a side view of the embodiment in FIG. 12;

FIG. 15 is a distal end view of the embodiment in FIG. 12;

FIG. 16 is a side view of the embodiment in FIG. 15, looking in the direction of arrows at section 16-16;

FIG. 17 is a view in perspective of the embodiment in FIG. 12 installed on a conduit;

FIG. 18 is a side view in cross-section of the embodiment in FIG. 17, also coupled to an atomizer and installed on a luer-lock end of a syringe;

FIG. 19 is a view in perspective of an alternative second locking element installed in registration on a conduit;

FIG. 20 is an exploded assembly view of the embodiment in FIG. 19;

FIG. 21 is a side view in cross-section of the embodiment in FIG. 20, taken at section 21-21;

FIG. 22 is an exploded assembly view in perspective of the embodiment in FIG. 19;

FIG. 23 is a distal end view of the embodiment in FIG. 19, taken from section 23-23 in FIG. 21, and looking in the direction of the arrows;

FIG. 24 is a proximal end view of the second locking element in FIG. 19; and

FIG. 25 is a plan view of a fiat sheet pattern that can be used to make the second locking element in FIG. 19.

### DETAILED DESCRIPTION OF THE ILLUSTRATED EMBODIMENTS

The present invention may be embodied to provide an automatically-disabled syringe assembly. At least certain embodiments permit a syringe to experience multiple filling and emptying cycles before the syringe is disabled subsequent to a final discharge cycle, typically using an end-use fluid-guiding device.

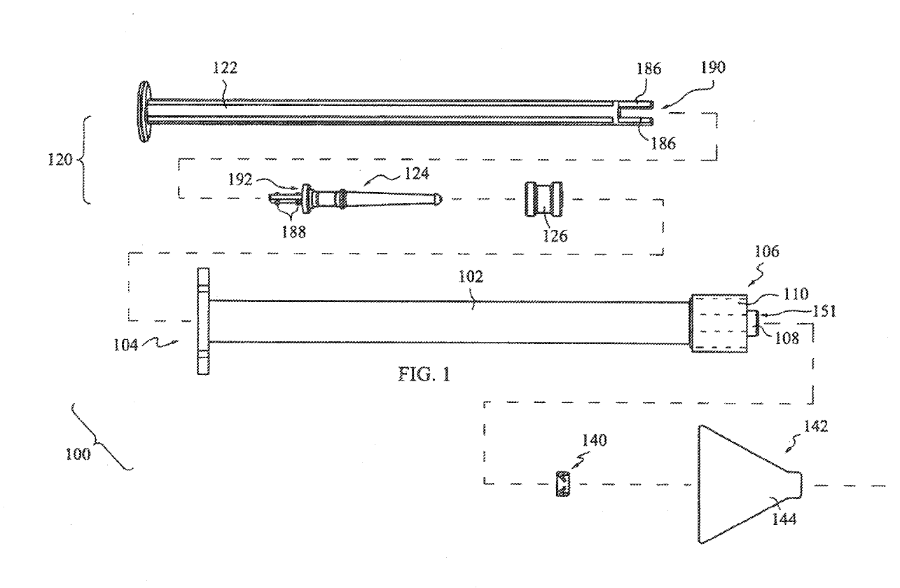

As illustrated in FIG. 1, one currently preferred embodiment is an assembly of a plurality of elements generally indicated at 100. Assembly 100 includes a syringe body 102 having an open proximal end, generally 104, and a distal end, generally 106. A syringe body 102 can be as large in fluid capacity as is desired. Syringe body 102 is illustrated in FIG. 1 as being substantially transparent. Of course, such is not required, and syringes having one or more partially opaque, or fully opaque, areas of construction are workable.

Distal end 106 of illustrated syringe body 102 carries luer-locking structure including a length of hollow male luer-locking tapered element 108, and a threaded female socket 110. The hollow portion of luer-locking element 108 forms a discharge orifice for the syringe body 102. Fluid-guiding attachments may be coupled in fluid-tight engagement with luer-locking element 108 in substantially conventional fashion. In the illustrated embodiment, internally threaded socket 110 is integrated into syringe body 102, forming a contiguous structure. In other workable embodiments, a socket 110 may be adapted to rotate with respect to the syringe body.

Although at least a short length of luer-locking taper 108 is highly desirable, alternative configurations are workable, including an externally-threaded hollow male discharge end (not illustrated). While the embodiment illustrated in FIG. 1 is based on the ubiquitous 6% conical taper luer-locking joint, principles of the invention apply to alternative structures, including alternative sizes of conventional luer-locking devices. The standard for 6% conical taper joints is set forth in ISO 594-2 Standard, “Conical Fittings with a 6% (Luer) taper for syringes, needles and certain other medical equipment—Part 2: Lock fittings”. The coupling conventionally present in an oral syringe (which is larger than the 6% luer-locking joint), is another alternative workable connector. A workable distal coupling structure of an alternative syringe body simply helps to form a third leak-resistant connection to a desired fluid-guiding attachment device or fluid dispensing or fluid acquisition.

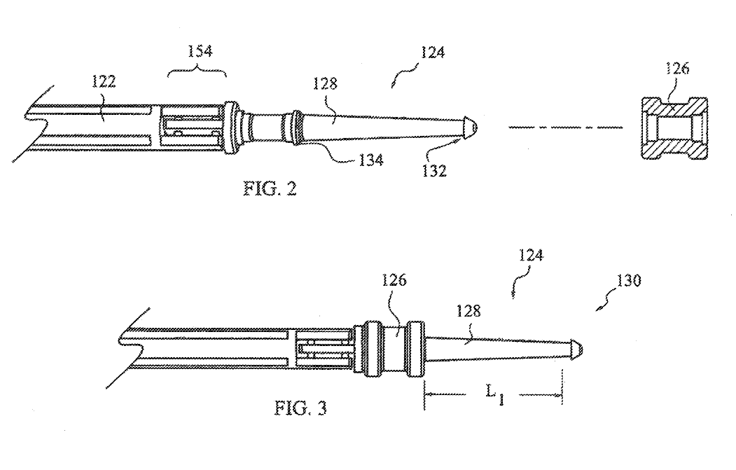

Still with reference to FIG. 1, assembly 100 also includes a plunger assembly, generally indicated at 120. Illustrated plunger assembly includes stem 122, core element, generally indicated at 124, and wiper element 126. Wiper element 126 is structured to be maintained in registration on core 124 and forms a fluid-tight seal for reciprocation inside body 102 to inspire and expel fluid from the assembly 100 (see also FIGS. 2 and 3). It is currently preferred, although not require, for core element 124 to be detachable from the stem 122. It should also be noted that alternatively structured stems may be used. For examples, the stem may alternatively include a thumb hole, or handle, to permit a user's thumb or hand, respectively, to reciprocate the stem.

As seen in FIGS. 2 and 3, a workable core 124 includes an elongate shaft 128 having an axial length in excess of L₁ and structured to dispose capture structure, generally indicated at 130, distal to the discharge aperture of a syringe body 102 when the plunger 120 is disposed at a fluid dose fully-expelled position. Capture structure 130 illustrated in FIGS. 2 and 3 includes a proximally facing surface, indicated generally at 132. By “proximally facing surface”, it is intended to encompass surfaces having a normal vector with a component directed in the proximal direction. In particular, the normal vector of a “proximally facing surface” is not required to be substantially parallel with a length axis of the core 124, as illustrated.

In certain embodiments, and as illustrated, a size and shape (e.g. diameter) of shaft 128 is desirably formed in relatively close agreement with the syringe discharge aperture (e.g. bore in hollow luer element 108) to reduce “dead space” and promote complete evacuation of the syringe's contents. While shaft 128 can typically reciprocate through the discharge aperture of syringe body 102 without interference, shoulder 134 of the illustrated embodiment forms a structural interference with body 102 when the plunger 120 is located at a fluid fully-expelled position. Shoulder 134 is an exemplary stopper element that resists travel of the core in a distal direction. In some cases, a wiper element, such as 126, can also function as a stopper.

For purpose of this disclosure, a locking element is effective to resist disassembly of two or more components of an assembly. With reference again to FIG. 1, assembly 100 further includes a first locking element, generally 140, and an (optional) fluid-dispensing or fluid-acquiring attachment, generally 142. A workable attachment 142 may include a fluid atomizer 144, although other conventional fluid-guiding structures or devices such as needles, tubing connectors, etc., are workable.

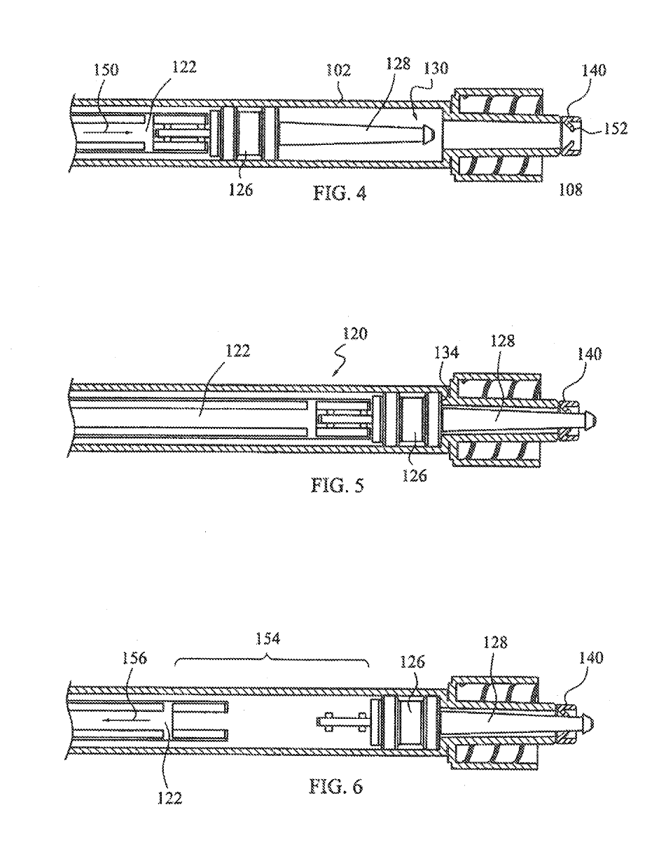

Principles of operation of an exemplary device structured according to certain principles of the invention are illustrated in FIGS. 4 through 6. In FIG. 4, stem 122 is being displaced in a distal direction from a dose-loaded position, as indicated by arrow 150, indicating that the plunger is being depressed and is moving in a fluid-expelling direction.

With particular reference to FIG. 4, an exemplary first locking element 140, which is a separate and distinct element from the syringe body 102, is disposed in registration at the distal end of the luer element 108. As illustrated, an exemplary first locking element 140 may be embodied essentially as a second length of hollow male luer-locking taper sized as an operable extension of a first length provided by luer-locking element 108.

A distal end of hollow male luer-locking taper 108 terminates at a distal interface, indicated generally at 151 in FIG. 1, that is illustrated as being disposed at about the plane of the distal end of socket 110. A proximal end of first locking element 140 includes a surface that is configured and arranged to cause a structural interference with the distal interface 151 effective to resist proximal displacement (from a captured position) of shaft 128 and capture structure 130.

FIG. 5 illustrates the plunger assembly 120 disposed at the fluid fully-expelled and captured position. Effectively, the discharge aperture of the syringe body 102 is pinned between an internally disposed stopper, such as shoulder 134, and an external capture structure, such as first locking element 140. Consequently, the plunger cannot reciprocate, and the syringe assembly is effectively rendered incapable of reuse. Note that exemplary capture structure 130 is engaged by cantilevered fingers 152 of exemplary first locking element 140. If first locking element 140 was not present, the capture structure 130 and distal end of shaft 128 would simply protrude from the discharge aperture at the distal end of luer element 108, and the plunger assembly 120 would be free to reciprocate.

It is desirable for capture structure to engage with locking structure without compromising usability of the syringe assembly. That is, with reference to FIGS. 4 and 5, it is preferred for the shaft 128 to engage with locking element 140 without requiring a user to exert an undue force on the stem 122. In certain cases, a user will be substantially unaware as the lock-up occurs.

FIG. 6 illustrates optional decoupling structure, generally indicated at 154, configured to permit separation of a distal end of plunger stem 122 from a proximal end of core element 124 responsive to a user attempting to displace the plunger assembly from a captured configuration (and in the proximal direction indicated by arrow 156). If a user were to retract the plunger stem 122 with sufficient force, decoupling structure 154 would permit stem 122 to separate from core 124. Core 124 consequently remains behind, and blocks the discharge aperture of the syringe, thereby effectively frustrating reuse of the syringe body 102.

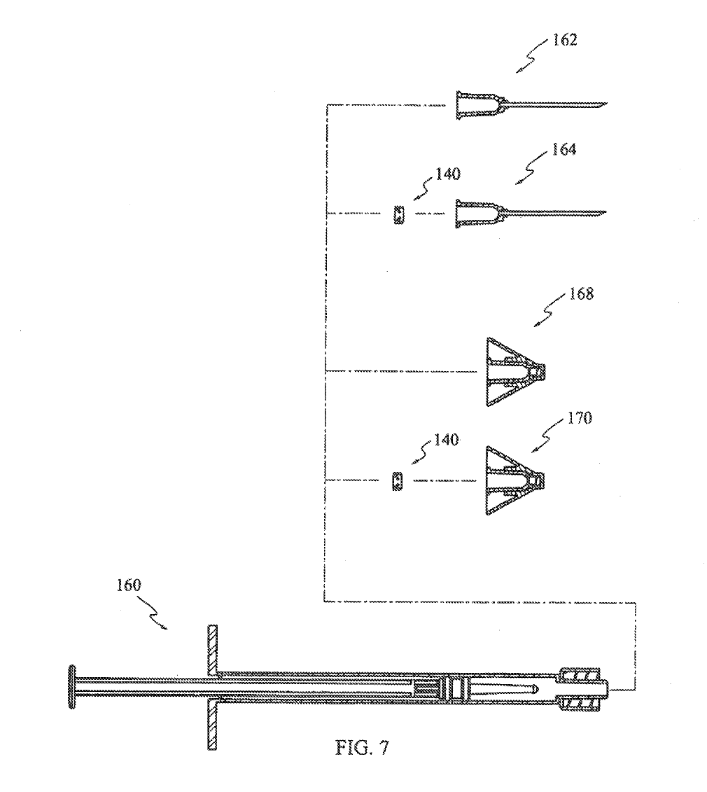

FIG. 7 illustrates a syringe, generally indicated at 160, that may be used in accordance with certain principles of the invention with a selected fluid guiding attachment. A non-limiting selection of fluid-guiding attachments are illustrated in FIG. 7. For example, a needle, generally indicated at 162, may be affixed to the distal end of syringe 160 to inspire fluid into the syringe. Then, the syringe 160 and needle 162 may be used to inject that inspired fluid into a container, e.g. to reconstitute a treatment substance. Needle 162 lacks a first locking element 140, so that syringe and needle combination can be reused a plurality of times. However, illustrated needle assembly 164 includes a first locking element 340 that is effective to resist proximal displacement of the plunger from a fluid fully-expelled position.

Similarly, syringe 160 may be reused a plurality of times with the atomizer generally indicated at 168. No structure is present in that illustrated combination effective to resist retraction of the plunger of syringe 160 from a fluid fully-expelled position. In contrast, the atomizer assembly indicated in 170 includes a first locking element 140 that is effective to resist proximal displacement of the plunger from a fluid fully-expelled position. Consequently, once the plunger of syringe 160 is captured in engagement with atomizer assembly 170, the syringe is effectively and automatically rendered incapable reuse.

Sometimes, a first locking assembly 140 is held in registration inside a fluid-guiding attachment, such as a needle assembly 164, or atomizer assembly 170 by way of a friction-fit, or press-fit. In certain other cases, a first locking element 140 can be a constituent part of, or permanently adhered to, or welded to, or otherwise affixed to, the fluid-guiding attachment. It is within contemplation that a locking element may even be positioned by alternative structure, such as a user's hand, to permit engagement of locking structure associated with the locking element with capture structure of a plunger.

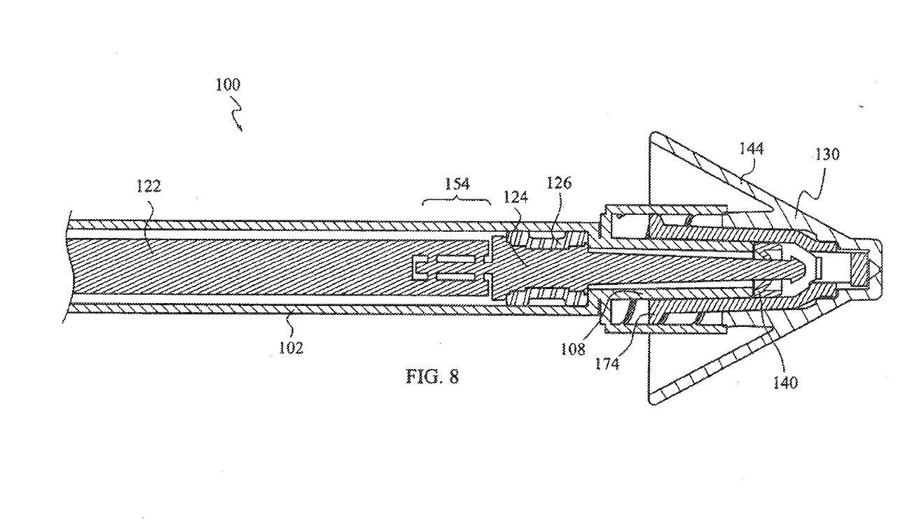

With reference now to FIG. 8, an exemplary assembly 100 is illustrated in a fluid fully-expelled and shaft-captured position. It can clearly be seen that exemplary first locking element 140, which is separate and discrete from syringe body 102, is disposable at a position distal to the syringe body. Exemplary first locking element 140 may sometimes be characterized as an operable extension of male lure-locking element 108 in that one operable embodiment of a first locking element 140 may be formed substantially as an extension of the male tapered luer-locking surface of luer element 108. Typically, locking element 140 is installed at a distally-spaced-apart operable location inside fluid-guiding structure, such as atomizer 144, to permit fluid-tight engagement of the outside luer-locking surface of male luer element 108 with the inside of the female luer element 174 before causing a structural interference between proximal structure of first locking element 140 and a distal interface 151 of luer element 108. Further, illustrated exemplary capture structure 130 is configured and arranged to couple with one or more cantilevered locking finger 152 carried at the proximal end of locking element 140, when stem 122 of the plunger assembly is displaced distally to the illustrated fully-expelled position. Such coupling is effective to resist retraction of the core 124, and frustrate reuse of the syringe assembly 100. In the event that a user pulls proximally on stem 122 with sufficient force, decoupling joint 154 would permit the stem 122 to separate from the core 124, and further frustrate reuse of the syringe assembly 100.

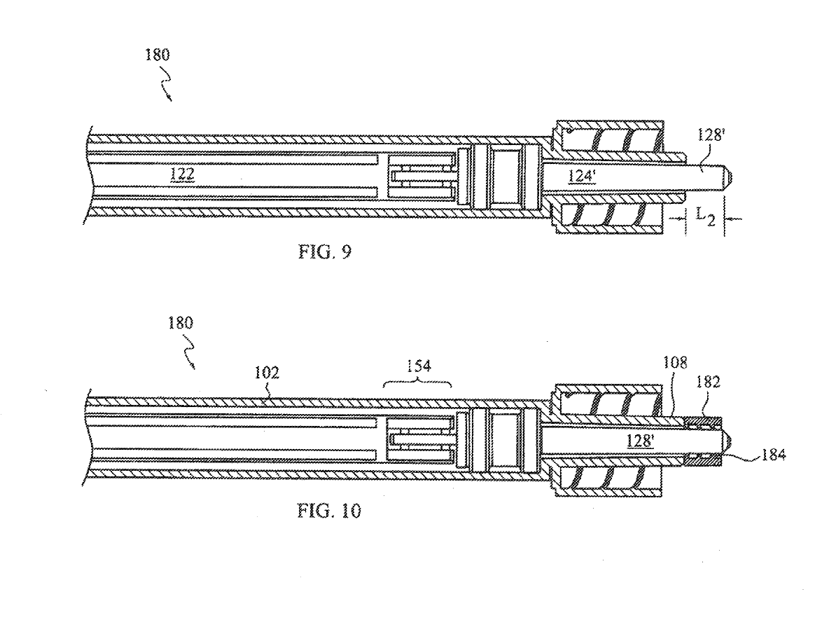

An alternative workable embodiment of an auto-disable syringe assembly structured according to certain principles of the invention is illustrated in FIGS. 9 and 10, and is generally indicated at 180. The extended shaft 128′ of assembly 180 is structured to essentially maximize expulsion of fluid from syringe body 102 and locking element 182. Such complete expulsion of fluid reduces waste of expensive treatment compounds.

It can be seen in FIG. 9 that exemplary extended shaft 128′ protrudes distally from syringe body 102, by an operable distance L₂, when at the illustrated fluid fully-expelled position. By “operable distance”, it is intended to mean that the protruding length L₂ is sufficient to permit application of an operable retaining force to the exposed portion of the shaft 128′. Known syringes adapted for such complete fluid expulsion (e.g. zero dead-space syringes) do not protrude a sufficient protruding length (if a protruding length is provided at all) to permit the required shaft loading.

A “retaining force” is defined, for purpose of this document, as resisting a user from pulling by hand on a stem 122 to retract the core element 124 from an entrapped position, such as illustrated in FIGS. 8 and 10. An exemplary “retaining force” keeps illustrated shaft 128 in a fluid-blocking position in the discharge opening of a syringe body, such as body 102. An “operable retaining force” will generally cause structural failure of a component or element before the plunger can retract from a captured position. In any case, an “operable retaining force” is sufficient to defeat a user from reusing the syringe assembly without resorting to tools and/or destroying one or more component.

It is recognized that certain luer-locking joints including a threaded interface can be rotated in an opening direction to generate a significant tensile force in, for example, a distally extending captured shaft. A first locking element that is bonded to, or integral with, a fluid-guiding device might well generate sufficient tensile force as to break a shaft 128 (see FIG. 3), and permit retraction of the plunger 122 from a captured position, and consequently, permit reuse of the syringe. Therefore, it is preferred, for the first locking element to be engaged to a fluid-guiding device by way of a friction-fit or pres-fit, that will inherently be decoupled or otherwise disengaged at a lower tensile load than that required to either break the shaft 128, or to compromise engagement between capture structure and a locking element. Consequently, although the captured first locking element may be disengaged from a fluid-guiding device as a user attempts to unscrew that fluid-guiding device, the first locking element will remain coupled to the capture structure and will resist reuse of the syringe by continuing to resist plunger reciprocation. Essentially, the press-fit coupling desirably forms a “weakest link” to prevent the plunger from breaking at a location that would permit reuse of the syringe.

The alternative first locking element 182 illustrated in FIG. 10 is configured to apply a retaining frictional force to the protruding length of extended shaft 128′. As illustrated, the surface of shaft 128′ includes a distally-oriented mold-release that and consequently, lacks a proximally facing surface. However, the plurality of self-biased fingers 184 are configured to apply a frictional force to shaft 128′ sufficient to resist proximal retraction of the syringe plunger. It is currently believed that a minimum operable distance L₂ to permit a friction-caused retaining force is at least about 1 times the diameter of an extended shaft 128′.

Components of an assembly, such as assembly 100, are typically injection molded. The various components are generally made from medical grade plastic, or plastic-like materials. It is currently preferred to fabricate a plunger stem 122 and detachable core 124 from polypropylene or ABS. A workable wiper element can be made from polyisoprene or non-latex polyisoprene. Typically, a syringe body 102 is made from polypropylene. A workable locking element, such as alternative first locking element 182 illustrated in FIG. 10, or first locking element 140 illustrated in FIG. 11, can be made from polycarbonate. Cantilevered and self-biased fingers (e.g. 184 or 152) can be embodied as a circumferentially-uninterrupted thread-like element, or a plurality of circumferentially-interrupted elements structured to permit removal of a locking element from an injection mold.

As mentioned above, it is preferred for the stem 122 to separate from the core 124 to further resist reuse of the syringe assembly 100. Many ways to calm such an effect will be apparent to designers of medical products. For example, a stem and core can be injection molded as a unitary piece, with a local area of reduced cross-section at a desired decoupling location being structured to fail (in shear or tension) before a critical cross-section of shaft 128 fails in tension and thereby permits retraction of the plunger.

As illustrated between FIGS. 1 and 2, the proximal end of core 124 is molded in registration between distally protruding legs 186 of the stem 122. Raised areas 188 may be configured with even farther reduced cross-section to reduce a shear-carrying area between the elements and facilitate decoupling. In any case, it is desirable for the pull-apart joint 154 to reliably remain together during even aggressive inspiration of fluid into the syringe, but then decouple relatively easily to automatically disable the syringe assembly from reuse.

With particular reference to FIG. 1, the distal end, generally 190, of stem 122 is structured in harmony with a compression bearing area 192 to ensure motion of core 124 in a distal direction responsive to stem displacement in that direction. Such compression interface ensures that sufficient force can be applied as required to engage locking structure and capture structure to effect an auto-destruct event.

In a currently preferred method of use, a user can inspire fluid into the syringe, then expel that fluid into a container to reconstitute a treatment substance. A needle may first be attached to the syringe to facilitate fluid inspiration and expulsion into a container, such as a vial having a pierceable top. The syringe can be fully emptied, meaning the plunger is completely depressed in a maximum distal, fluid fully-expelled position. Treatment substance may then be inspired into the syringe by retracting the plunger stem. The user may then couple the syringe with a fluid-guiding attachment that includes a first locking structure. Until first locking structure is affixed to the syringe plunger, the syringe can be fully emptied and re-filled a plurality of times. An operable fluid-guidance attachment includes a fluid atomizer, such as may be used for nasal therapy. One or more dose of treatment substance may be expelled until the syringe is empty. The user will auto-disable the syringe upon dispensing a dose to empty the syringe. Sometimes, subsequent to capturing structure of the plunger with a first locking element at a position distal to the discharge of the syringe body, the user may pull proximally on the plunger stem to cause separation of a proximal stem portion, leaving behind proximally untethered obstruction disposed in penetration through the syringe discharge aperture to resist reuse of the syringe.

Sometimes, a second locking element may be included in certain embodiments of the inventions. Certain of such second locking elements may also be used as a stand-alone looking element. In any case, a second locking element is effective to resist removing a conduit from an installed position in engagement with a conventional luer-lock device. A currently preferred embodiment resists rotation (in a loosening, or disassembly direction), of circumferentially spaced apart male threads from an installed position inside a female thread, such as is carried at the distal end of many fluid-dispensing syringes.

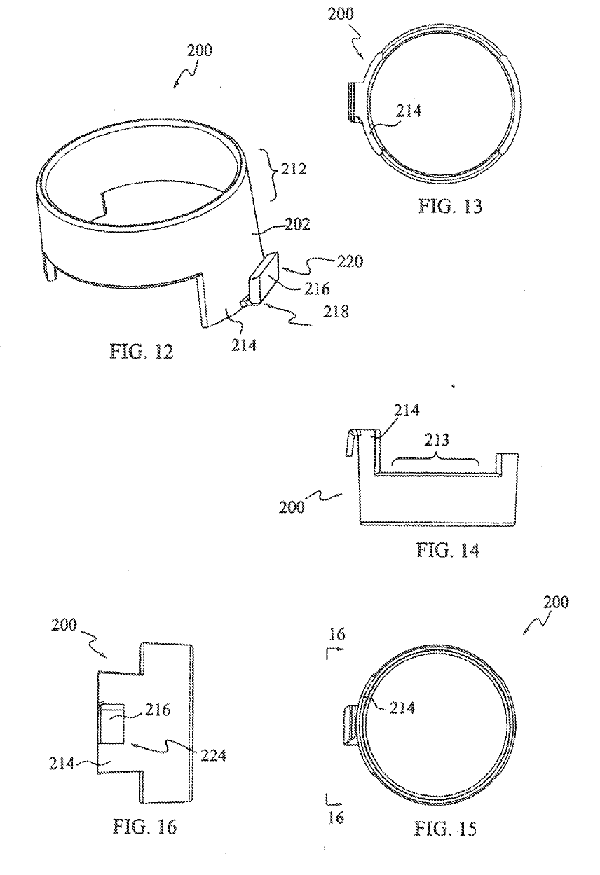

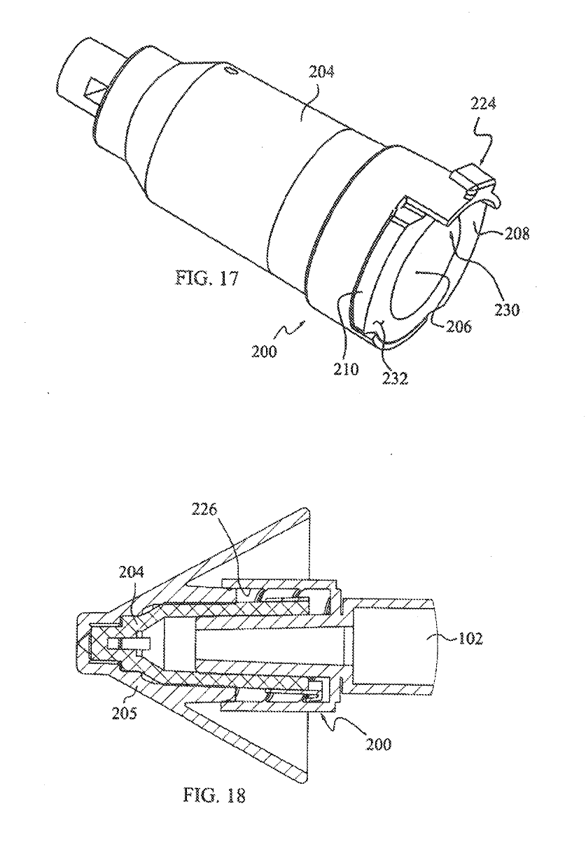

FIGS. 12 through 18 illustrate structural details of a first currently preferred embodiment of a second looking element, generally indicated at 200. Second locking element 200 includes an arcuate wall 202 configured for disposition in engagement around the external surface of as generally cylindrical conduit element. An illustrative conduit element includes hub 204 in FIG. 17. Hub 204 is a portion of a fluid atomizer 205 (see FIG. 18). A wide range in alternative conduit structures may be used in combination with certain embodiments of second locking elements according to certain principles of the invention. Non-limiting examples of Workable conduit structures include: needle hubs, conduit connectors, fluid atomizers, and the like. A second locking element may be used, for example, to resist removal of fluid-guiding structure from a fixed and permanent attachment to a syringe.

Hub 204 is configured to interface with conventional luer-lock structure of a luer-lock device, such as is carried at the dispensing (distal) end of certain fluid-dispensing syringes. Hub 204 includes an internal bore 206 configured to couple in fluid-tight engagement with a male luer of the conventional luer-lock device. A proximal end of hub 204 carries circumferentially interrupted male threads, 208 and 210, respectively, that are structured to couple with a female thread of the luer-lock device.

Wall 202 includes a band portion, generally 212, configured to wrap around a sufficient portion of the circumference of conduit 204 to resist radial separation between arcuate wall 202 and conduit 204. The illustrated band portion 212 is configured to wrap around more than half the circumference of hub 204. An alternative configuration within contemplation includes a proximal surface having a through-hole to permit engagement of a male luer inside bore 206, and a pair of distally projecting arms that together form an alternatively configured circumferentially interrupted wall.

Desirably, band 212 includes at least a first blocking portion, generally 213 in FIG. 14, forming a structural interference with at least one of male threads 208, 210 to resist axial displacement of the wall 202 in a proximal direction with respect to conduit 204. At least one proximally projecting tang portion 214 of wall 202 is sized to fit into a circumferential space between the male threads 208, 210 and thereby cause a structural interference with one of threads 208, 210 to resist rotation of an installed wall 202 about the centerline of bore 206.

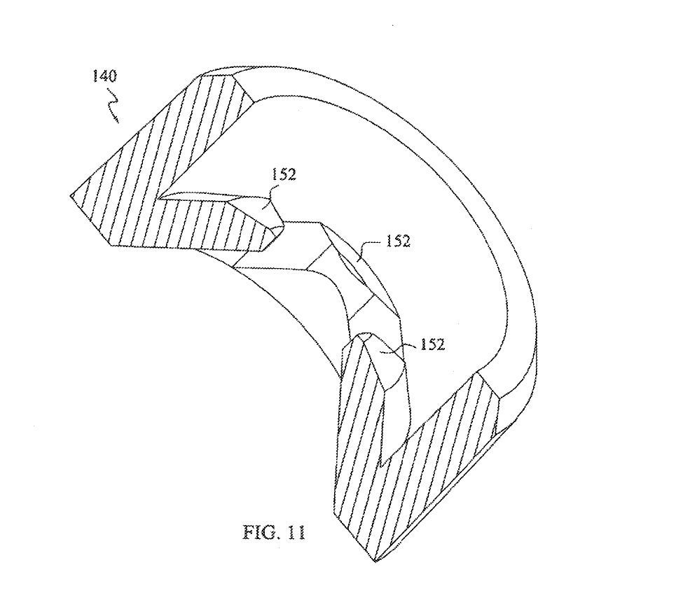

Wall 202 also carries a flap 216 with a leading edge generally indicated at 218, and a trailing edge generally indicated at 220. Desirably, leading edge 218 is configured to facilitate rotation of all 202 in a tightening direction when assembly is installed in a luer-lock device. Illustrated leading edge 218 is radiused and disposed in sufficient radial proximity to the surface 222 as to rotatably fit into the female thread of a luer-lock device without interference. In contrast, flap 216 includes a cantilevered portion extending to the trailing edge 220 that is configured to develop a self-bias as male threads 208, 210 are rotated in a tightening direction with respect to the female thread of the luer-lock device. Further, the trailing edge 220 is configured to cause a structural interference with the female thread to resist rotation of male threads 208, 210 in a loosening direction. As illustrated, trailing edge 220 may be sharpened. Trailing edge 220 may also be twisted to cause a corner, generally indicated at 224 in FIGS. 16 and 17, to preferentially contact, and dig into, the material forming the female thread (e.g. 226 in FIG. 18 of a luer-lock device. A desirable twist in flap 216 also facilitates entrance of the proximal end of hub 204 into the opening of the female thread of luer-lock device.

It is within contemplation that a second locking element structured according to certain principles of the invention may include one or more second blocking structure to resist displacement of an installed locking element in a distal direction. For example, the corner generally indicated at 230 in FIG. 17 may be bent radially inward subsequent to installation of locking element 200 onto hub 204. Alternative structure may be provided to essentially form a finger carried at a proximal end of wall 202, with the finger projecting radially inward to cause a structural interference with a proximally facing surface, such as surface 232, of hub 204 to resist axial displacement of an installed 202 in a distal direction with respect to the hub 204.

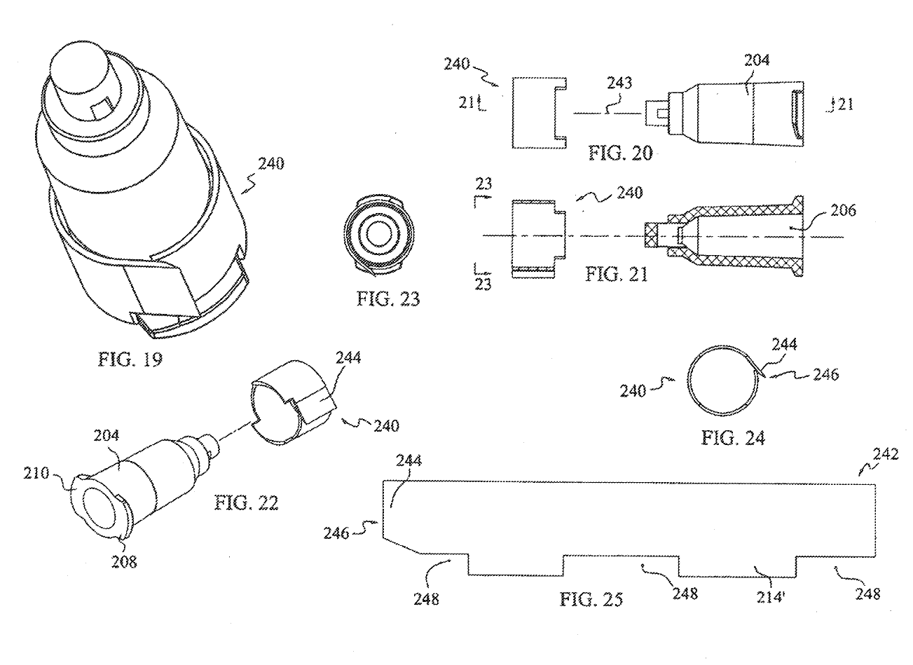

A second embodiment of a workable second locking element is illustrated in FIGS. 19 through 25, and is generally indicated at 240. An exemplary second locking element 240 may be made by cutting the plan form generally indicated at 242 in FIG. 25 from thin Stainless Steel sheet stock. The plan form 242 may then be rolled-up around an axis 243 to form a generally cylindrical section having a cantilevered tongue 244. The tip of the tongue 244, generally 246, may be sharpened, or even arranged to have a point, to facilitate making biting contact with a female thread 226. A tang 214′ is sized to be received between male threads 208, 210. A void 248 is sized to receive a male thread 208 or 210.

Desirably, a second locking element is made from metal, such as Stainless Steel. Thin Stainless Steel sheet stock advantageously can fit into the relatively small available radial space in a luer-lock device. Also, such material is strong enough to resist undesired rotation of components without experiencing structural failure. Further, such material can be configured to “bite” well into the (typically) plastic female thread of a luer-lock device to form a structural interference,.

What is claimed is:

1. An apparatus, comprising:
a syringe body extending from an open proximal end to a discharge aperture at a distal end;
a plunger amenably disposable for reciprocal motion inside said body between a dose-loaded position and a fully-expelled position, a distal end of said plunger assembly carrying capture structure configured to protrude distally, by an operable distance, from said discharge aperture when said plunger assembly is disposed at said fully-expelled position.

2. The apparatus according to claim 1, further comprising:
a first locking element that is separate and discrete from said syringe body and disposable at a position distal to said distal end of said syringe body, said first locking element being configured and arranged to couple with said capture structure, when said plunger is displaced distally to said fully-expelled position, effective to resist reuse of said syringe.

3. The apparatus according to claim 1, wherein:
said plunger assembly comprises decoupling structure configured to permit separation of a proximal stem end of said plunger assembly from said distal end of said plunger assembly responsive to a user attempting to proximally displace said planer assembly from a captured configuration comprising said fully-expelled position.

4. The apparatus according to claim 2, wherein:
said distal end of said syringe body carries a length of hollow male luer-locking taper terminating at a distal interface; and
a proximal end of said first locking element is configured and arranged to cause a structural interference with said distal interface effective to resist proximal displacement of said capture structure.

5. The apparatus according to claim 2, wherein:
said capture structure comprises a proximally facing surface; and
locking structure of said first locking element is configured and arranged to cause a structural interference with said proximally facing surface.

6. The apparatus according to claim 5, wherein:
said locking structure comprises a self-biased finger.

7. The apparatus according to claim 2, wherein:
locking structure of said first locking element comprises a plurality of self-biased fingers that are displaceable by proximal movement of said capture structure to form a friction contact interface between said fingers and said capture structure.

8. The apparatus according to claim 2, wherein:
said distal end of said syringe body carries a first length of hollow male luer-locking taper terminating at a distal interface; and
said first locking element comprises a second length of hollow male luer-locking taper sized as an operable extension of said first length.

9. The apparatus according to claim 3, wherein:
said decoupling structure comprises a pull-apart joint disposed at an intermediate location of said plunger assembly, said pull-apart joint comprising a first interface structured to fail in shear under a lower tension load than required for a critical cross-section of said capture structure to fail under tension.

10. The apparatus according to claim 9, wherein:
said pull-apart joint comprises one-way pull-apart joint with mutually cooperating surfaces of said plunger assembly being structured to bear in compression as said plunger is displaced from said dose-loaded position toward said fully-expelled position.

11. The apparatus according to claim 2, wherein:
said locking element is affixed to a fluid-dispensing or fluid-acquiring attachment. said attachment being structured to permit corresponding ejection or inspiration of fluid through said attachment by operation of said plunger.

12. The apparatus according to claim 2, wherein;
said first locking element is an integral part of a fluid-dispensing or fluid-acquiring attachment, said attachment being structured to permit corresponding ejection or inspiration of fluid through said attachment by operation of said plunger.

13. An auto-disable syringe having a plunger disposed for reciprocation of a stopper inside a body effective to inspire and eject fluid, the improvement comprising:
a first locking element that is separate and discrete from said body and disposable at an operable position at a distal end of said body, said first locking element being configured and arranged to couple with capture structure carried by said plunger, when said plunger is displaced distally to a fluid fully-expelled position, effective to resist proximal displacement of said stopper and thereby frustrate reuse of said syringe.

14. The syringe according to claim 13, wherein:
said first locking element is affixed to a fluid-dispensing or fluid-acquiring attachment, said attachment being structured to permit corresponding ejection, or inspiration of fluid through said attachment by operation of said plunger.

15. The syringe according to claim 13, wherein:
said first locking element is an integral part of a fluid-dispensing or fluid-acquiring attachment said attachment being structured to permit corresponding ejection or inspiration of fluid through said attachment by operation of said plunger,

16. The syringe according to claim 13, wherein:
said first locking element is structured in harmony with a cooperating fluid-guiding desire to form a friction coupling with said device, said friction coupling being effective to permit ejection of fluid through said device by operation of said plunger and to operate as a weakest link to resist damage to a distally extending shaft of said plunger.

17. A method, comprising;
providing an auto-disable syringe having a plunger disposed for reciprocation in a body effective to inspire and eject fluid;
coupling a fluid-inspiring device to said syringe;
inspiring a dose of fluid into said syringe;
injecting at least a portion of said dose of fluid into a container effective to reconstitute a treatment substance;
displacing said plunger to a fluid fully-expelled position;
inspiring a portion of said treatment substance into said syringe; and
distally displacing said plunger to said fluid fully-expelled position to expel said portion while simultaneously coupling a first locking element to said plunger effective to resist retraction of said plunger from said fully-expelled position.

18. The method according to claim 17, further comprising:
after inspiring a portion of said treatment substance into said syringe, coupling a fluid-dispensing device to said syringe, said fluid-dispensing device carrying said first locking element, said first locking element being structured to couple with capture structure carried by said plunger.

19. The method according to claim 18, wherein
said capture structure is configured to protrude distally from a discharge aperture of said syringe when said plunger is disposed at said fluid fully-expelled position.

20. The method according to claim 17, further comprising:
subsequent to coupling said first locking element to said plunger, proximally retracting a stem portion of said plunger effective to separate said stem portion flora a distal portion of said plunger.

21. In a device including a generally cylindrical length of conduit having an internal bore configured to couple in fluid-tight engagement with a male luer of a conventional luer-lock device, a proximal end of the conduit carrying circumferentially interrupted male threads that are structured to couple with a female thread of the luer-lock device, the improvement comprising:
an arcuate wall configured for disposition in engagement around the external surface of said conduit;
a hand portion of said wall being configured to wrap around a sufficient portion of the circumference of said conduit to resist radial separation between said arcuate wall and said conduit, a portion of said band forming a structural interference with at least one of said male threads to resist axial displacement of said wall in a proximal direction with react to said conduit;
a proximally projecting tang portion of said wall being sized to fit into a circumferential space between said male threads and thereby cause a structural interference to resist rotation of said wall about the centerline of said bore; and
a flap carried by said wall, a cantilevered portion of said flap being configured to develop a self-bias as said male threads are rotated in a tightening direction with respect to said female thread, a trailing edge of said flap being configured to cause a structural interference with said female thread to resist rotation of said male threads in a loosening direction.

22. The improvement according to claim 21, wherein:
said trailing edge is sharpened.

23. The improvement according to claim 21, wherein:
the leading edge of said flap is configured to facilitate rotation of said wall in said tightening direction.

24. The improvement according to claim 21, further comprising:
a finger carried at a proximal end of said wall, said finger projecting radially inward to cause a structural interference with a proximally facing surface of said conduit to resist axial displacement of said wall in a distal direction with respect to said conduit.

25. The improvement according to claim 21, wherein:
said band portion is configured to wrap around more than half the circumference of said conduit.
